(19) United States
(12) Patent Application Publication
(10) Pub. No.: US 2024/0001134 A1
(43) Pub. Date: Jan. 4, 2024

(54) ADHESIVE LAYER WITH PROTECTIVE BORDER FOR USE IN A TUMOR TREATING FIELDS TRANSDUCER

(71) Applicant: Novocure GmbH, Root D4 (CH)

(72) Inventors: Yoram WASSERMAN, Haifa (IL); Stas OBUCHOVSKY, Haifa (IL); Nataliya KUPLENNIK, Haifa (IL); David SHAPIRO, Haifa (IL)

(73) Assignee: Novocure GmbH, Root D4 (CH)

(21) Appl. No.: 18/216,258

(22) Filed: Jun. 29, 2023

Related U.S. Application Data
(60) Provisional application No. 63/357,278, filed on Jun. 30, 2022.
(60) Provisional application No. 63/357,390, filed on Jun. 30, 2022.
(60) Provisional application No. 63/408,604, filed on Sep. 21, 2022.
(60) Provisional application No. 63/420,950, filed on Oct. 31, 2022.
(60) Provisional application No. 63/421,005, filed on Oct. 31, 2022.

(51) Int. Cl. A61N 1/40; H01B 1/24; A61L 24/02; A61L 24/00; A61K 9/00

(52) U.S. Cl. CPC A61N 1/40; H01B 1/24; A61L 24/02; A61L 24/001; A61L 24/0078; A61K 9/0009; A61L 2400/00

(57) ABSTRACT

An adhesive layer for use in a transducer apparatus, the adhesive layer extending in an x-y plane and having an adhesive layer outer edge, the adhesive layer including: an adhesive matrix material; a plurality of electrically conductive particles embedded at least partially within the adhesive matrix material forming a conductive adhesive region of the adhesive layer; and at least one non-conductive edge portion comprising an adhesive devoid of electrically conductive particles, the at least one non-conductive edge portion being electrically non-conductive; wherein, when viewed in a direction perpendicular to the x-y plane, a first non-conductive edge portion is located adjacent to and extends along an outer edge of the conductive adhesive region and forms at least a portion of an outer perimeter of the adhesive layer.

### CROSS-REFERENCE TO RELATED APPLICATIONS

The present application claims the benefit of and priority to U.S. Provisional Patent Application No. 63/357,278, filed Jun. 30, 2022; U.S. Provisional Patent Application No. 63/357,390, filed Jun. 30, 2022; U.S. Provisional Patent Application No. 63/408,604, filed Sep. 21, 2022; U.S. Provisional Patent Application No. 63/420,950, filed Oct. 31, 2022; and U.S. Provisional Patent Application No. 63/421,005, filed Oct. 31, 2022, the contents of each of which are incorporated herein by reference in their entireties.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into the region of interest by transducers placed on the patient's body and applying AC voltages between the transducers. Conventionally, transducers used to generate TTFields include a plurality of electrode elements comprising ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include arrays of ceramic disks attached to the subject's body via a conductive skin-contact layer such as a hydrogel.

### BRIEF DESCRIPTION OF THE DRAWINGS

FIGS. 1A and 1B depict an example transducer with an adhesive layer.

FIGS. 2A-2C depict another example transducer with an adhesive layer.

FIGS. 3A-3E depict example adhesive layers having a non-conductive edge.

FIGS. 4A-4D depict other example adhesive layers having a non-conductive edge.

FIG. 5 depicts example arrangements of transducers located on a subject's head.

### DESCRIPTION OF EMBODIMENTS

This application describes exemplary transducer apparatuses used to apply TTFields to a subject's body for treating one or more cancers.

Transducers used to apply TTFields to a subject's body often include multiple electrode elements coupled together on a substrate and attached to the subject's body at a desired location, for example, via an adhesive layer on the substrate or a separately applied adhesive. Transducers can include one or more conductive material layers located between the electrode elements and the subject's body upon attachment of the transducer to the subject's body. Such conductive material layers may include, for example, a conductive skin-contact layer such as a hydrogel or a conductive adhesive layer located against the subject's body. The conductive adhesive layer may take the form of an adhesive matrix material having conductive particles (e.g., carbon fibers or carbon black powder) embedded at least partially in the adhesive matrix material. Additionally, the conductive material layer(s) may include a conductive layer of anisotropic material taking the form of a carbon layer, a graphite layer, or others. The conductive layer of anisotropic material may have different thermal and/or electrical conductivities in a direction perpendicular to a face of the transducer (z-direction) than in directions parallel to the transducer face (directions in an x-y plane). Conductive material layer(s) having greater thermal conductivity in the x-y plane than in the z-direction can spread out heat generated by the electrode elements within an x-y plane while conducting electricity from the electrode elements in a z-direction toward the subject's body. This allows greater currents to be applied to the electrode elements while maintaining the temperature at the subject's skin under a maximum operating temperature.

In general, one or more pairs of transducers are positioned on the subject's body and used to alternately apply TTFields to the subject's body. Generally, it is preferred that there are at least two pairs of transducers, and that the transducers are not touching each other. However, on certain areas (e.g., the head) of the subject's body, two or more transducers may be positioned such that they overlap one another or are located immediately adjacent one another. It is important to avoid electrical contact between overlapping/adjacent transducers, particularly those with conductive material layer(s) that are highly conductive in the x-y plane, so as to prevent a short circuit in which current runs through the transducers and not through the subject's body. Electrical contact between transducers is typically avoided via physical separation of the conductive elements of the transducers. For example, physical separation is provided by a non-conductive adhesive bandage provided on each transducer, the adhesive bandage extending in the x-y plane beyond the outer edges of the electrode elements and any conductive material layers.

However, subjects will sometimes cut the adhesive bandage of a transducer, either to resize the transducer for fitting on a portion of the subject's body or to reduce the total contact area of the adhesive bandage (which can cause skin irritation) with the subject's body. Cutting the bandage in this manner could lead to the unintentional and undesirable exposure of a conductive material layer. Exposure of a conductive material layer of the transducer may eliminate the physical separation of conductive elements between adjacent transducers, potentially causing a short circuit between the transducers.

The inventors have now recognized that a need exists for transducers capable of preventing physical exposure of conductive material layer(s) of the transducer to a conductive portion of a nearby transducer.

Exemplary transducer apparatuses include a conductive adhesive layer having at least one non-conductive edge portion that is electrically non-conductive. The non-conductive edge portion may prevent exposure of the edges of a conductive portion (e.g., a conductive portion of the adhesive layer and/or a conductive layer of anisotropic material) of the transducer and, as such, may prevent two transducers from electrically connecting.

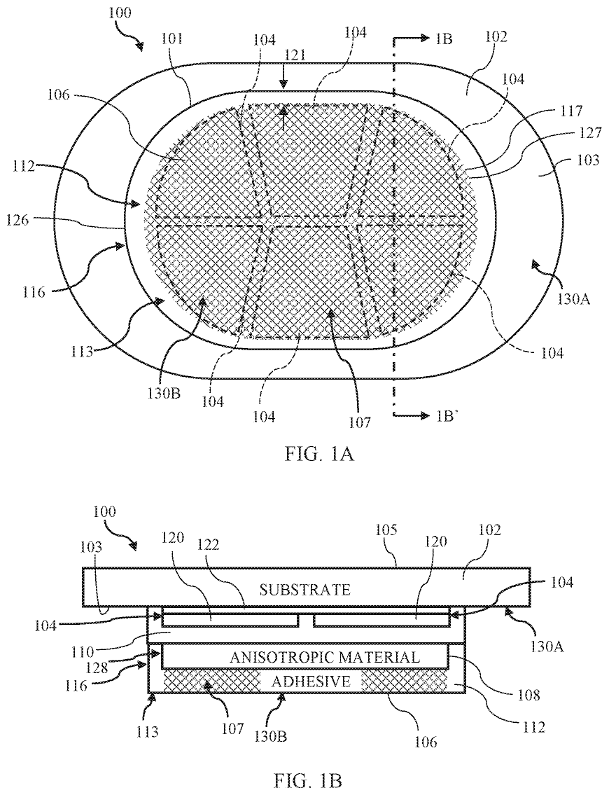

FIGS. 1A and 1B depict an example transducer 100 with an adhesive layer 106 having a non-conductive edge portion. FIG. 1A is a bottom view showing the front face of the transducer 100, and FIG. 1B is a side cross-sectional view of the transducer 100 (the cross-section as shown in FIG. 1A by the dashed line 1B-1B′).

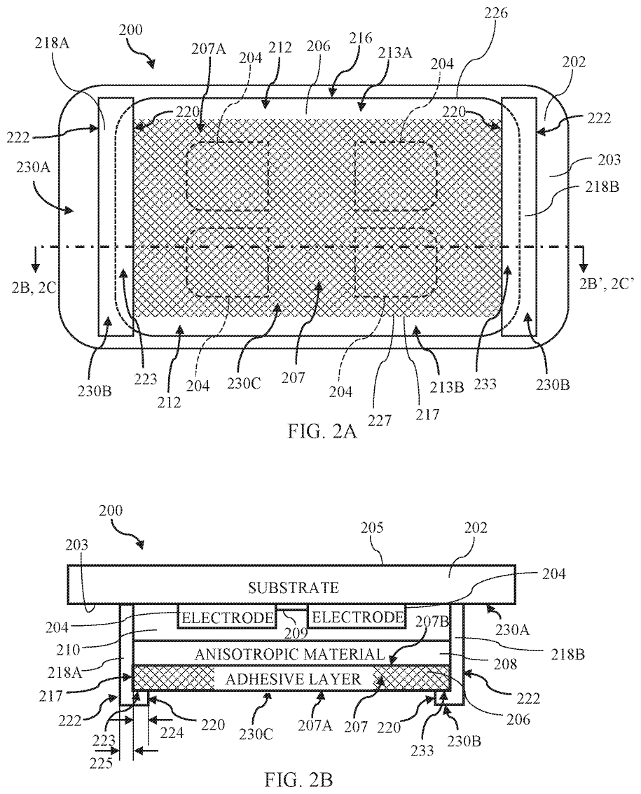

FIGS. 2A-2C depict another example transducer 200 with an adhesive layer 206 having a non-conductive edge portion. FIG. 2A is a bottom view showing the front face of the transducer 200, and FIGS. 2B and 2C are two examples of a side cross-sectional view of the transducer 200 (the cross-section as shown in FIG. 2A by the dashed line 2B, 2C-2B′, 2C′).

Each transducer (100, 200) of FIGS. 1A-2C is capable of delivering tumor treating fields to a subject's body.

In FIGS. 1A-2C, the transducer (100, 200) includes a substrate (102, 202), at least one electrode element (104, 204) coupled to the substrate (102, 202), and an adhesive layer (106, 206) comprising one or more electrically conductive adhesive regions (107, 207) electrically coupled to the at least one electrode element (104, 204). The substrate (102, 202) has a front face (103, 203) and a back face (105, 205), and the electrode element(s) (104, 204) are located on a side of the front face (103, 203) of the substrate (102, 202). As illustrated, the adhesive layer (106, 206) is located on an opposite side of the electrode element(s) (104, 204) from the substrate (102, 202).

The transducer (100, 200) of each of FIGS. 1A-2C may be affixed to the subject's body via the substrate (102, 202). Suitable materials for the substrate (102, 202) may include, for example, cloth, foam, flexible plastic, and/or a conductive medical gel or adhesive.

In FIGS. 1A-2C, the transducers (100, 200) comprise arrays of substantially flat electrode element(s) (104, 204). For each figure, the array of electrode elements (104, 204) may be capacitively coupled. In one example, as shown in FIG. 1B, the electrode elements 104 are non-ceramic dielectric materials 120 positioned over a plurality of flat conductors 122 (or a shared flat conductor 122 as shown in FIG. 1B). When viewed in a direction perpendicular to the face of the transducer, the non-ceramic electrode elements may take any desired shape (e.g., elements 104 in FIG. 1A). Examples of non-ceramic dielectric materials 120 positioned over flat conductors 122 include polymer films disposed over pads on a printed circuit board or over substantially planar pieces of metal. Preferably, such polymer films have a high dielectric constant, for example having a dielectric constant greater than 10. In another example, as shown in FIGS. 2B and 2C, the electrode elements 204 are ceramic electrode elements coupled to each other via conductive wiring 209. When viewed in a direction perpendicular to the face of the transducer, the ceramic electrode elements may be circular shaped or non-circular shaped (e.g., 204 in FIG. 2A). In other embodiments, the array of electrode elements (104, 204) are not capacitively coupled, and there is no dielectric material (such as ceramic, or high dielectric polymer layer) associated with the electrode elements (104, 204). The electrode elements (104, 204) may take any of these forms without departing from the scope of the present disclosure.

The adhesive layer (106, 206) may take any desired shape. For example, as shown in FIG. 1A, an outer perimeter 116 of the adhesive layer 106 may have a circular, oval, ovoid, ovaloid, or elliptical shape. As another example, as shown in FIG. 2A, an outer perimeter 216 of the adhesive layer 206 may have a substantially square or rectangular shape, or substantially square or rectangular shape with rounded corners. In FIGS. 1A and 2A, the outer perimeter (116, 216) of the adhesive layer (106, 206) defines an areal footprint (126, 226) of the adhesive layer (106, 206). Also, the conductive adhesive region (107, 207) has an outer perimeter (117, 217) which defines an areal footprint (127, 227) of the conductive adhesive region (107, 207). As illustrated, the areal footprint (127, 227) of the conductive adhesive region (107, 207) overlays one, or more than one, electrode element (104, 204). The conductive adhesive region (107, 207) may take any desired shape. For example, and as shown in FIG. 1A, it may have a circular, oval, ovoid, ovaloid, or elliptical shape. As another example, and as shown in FIG. 2A, it may have a substantially square or rectangular shape, or substantially square or rectangular shape with rounded corners.

The conductive adhesive region (107, 207) of the adhesive layer (106, 206) may be a composite adhesive layer. In particular, the conductive adhesive region (107, 207) comprises a plurality of electrically conductive particles embedded at least partially within an adhesive matrix material. The electrically conductive particles may include carbon granules, carbon flakes, graphite powder, carbon black powder, carbon nanoparticles, carbon nanotubes, and the like. The electrically conductive particles may include electrically conductive fibers, such as carbon fibers, as discussed in detail below. The electrically conductive fibers are illustrated in FIGS. 1A-2C via a mesh pattern covering part of the areal footprint of the adhesive layer (106, 206), which corresponds to the conductive adhesive region (107, 207).

As viewed in FIGS. 1A and 2A, the adhesive layer (106, 206) has at least two distinct areal regions: a first region (a non-conductive region, e.g., “non-fiber region”) (112, 212) that is devoid of electrically conductive particles, and a second region (conductive adhesive region, e.g., “fiber region”) (107, 207) having electrically conductive particles disposed therein, and having an outer edge (117, 217) of the conductive adhesive region (107, 207). The first region (112, 212) includes one or more areas (113, 213A/B) defining at least a first portion of the outer perimeter (116, 216) of the adhesive layer (106, 206). In some embodiments of this aspect, and elsewhere herein, the matrix material (e.g., adhesive) may be the same in each of the non-conductive region and the conductive adhesive region. In other embodiments of this aspect, and elsewhere herein, the matrix material (e.g., adhesive) may not be the same in each of the non-conductive region and the conductive adhesive region.

As shown in FIGS. 1A and 1B, the area 113 of the adhesive layer 106 that is devoid of electrically conductive particles may extend along the entire outer perimeter 116 of the adhesive layer 106. That is, the area 113 may define the entire outer perimeter 116 of the adhesive layer 106. This area 113 may be generally ring-shaped, annular shaped, or otherwise shaped like a continuous border when viewed from a direction perpendicular to the front face 103 of the substrate 102. In such embodiments, no electrically conductive particles are present along the entire outer perimeter 116 of the adhesive layer 106, and the adhesive layer 106 provides a built-in “skirt” of material without electrically conductive particles. This non-conductive edge portion 113 may extend a distance 121 of at least 1 mm, at least 2 mm, at least 3 mm, or more, from the adhesive layer outer edge 101 to the outer edge 117 of the conductive adhesive region 107 in a direction perpendicular to the adhesive layer outer edge 101. In some embodiments the distance 121 may be approximately constant around the whole perimeter, but in other embodiments the one or more areas 113 may present a varying distance 121 from the adhesive layer outer edge 101 to the outer edge 117 of the conductive adhesive region 107.

The area 113 in FIGS. 1A and 1B acts as a protective border that is entirely built into the adhesive layer 106. The adhesive layer 106 may be manufactured such that the second region 107 containing the electrically conductive particles is surrounded on all sides by this area 113 devoid of electrically conductive particles.

In other embodiments, as shown in FIGS. 2A-2C, the adhesive layer 206 may be manufactured such that fewer than all sides of the second region 207 containing the electrically conductive particles are surrounded by the area(s) 213A/B devoid of electrically conductive particles. This may be the case, for example, if the adhesive layer 206 is manufactured by rolling out or coating (for example, coating on a high speed adhesive coating line) with the areas 213A/B devoid of conductive particles on two opposing sides of the layer and subsequently cut to fit on the substrate 202. Alternatively, this can be accomplished by coating the same roll of substrate twice (e.g., a roll of substrate can run through a coating line such, that on a first pass, the center portion of the substrate is coated with the electrically conductive adhesive, which is then dried in the coating line oven(s) before being collected on a collection roll; the latter is then utilized as the substrate roll in a second coating using a non-conductive adhesive and shims at the coating head that control application of the non-conductive adhesive only on the two outside edges of the substrate adjacent to, or overlapping the edges of, the central coating of the electrically conductive adhesive.

As shown in FIGS. 2A-2C, the second region 207 (having electrically conductive particles) may include area(s) defining at least a portion of the outer perimeter 216 of the adhesive layer 206. For example, the second region 207 may define two opposite facing portions of the outer perimeter 216 of the adhesive layer 206. In FIGS. 2A-2C, a second area 223 defining a second portion of the outer perimeter 216 has electrically conductive particles, and a third area 233 defining a third portion of the outer perimeter 216 opposite the second portion of the outer perimeter 216 has electrically conductive particles.

As shown in FIGS. 2A-2C, the transducer 200 may include a non-conductive material border 218A (in FIG. 2A, 2B), 218A′ (in FIG. 2C) disposed over the second area 223. In addition, the transducer 200 may include a second non-conductive material border 218B, 218B′ disposed over the third area 233. The non-conductive material borders 218A, 218B, 218A′, 218B′ are electrically non-conductive. Two non-conductive material borders 218A/218B in FIG. 2B can be used with the transducer 200 in FIG. 2A, and two non-conductive material borders 218A′/218B′ in FIG. 2C can be used with the transducer 200 in FIG. 2A. However, other numbers and arrangements of such non-conductive material borders may be used to cover edge areas of the conductive adhesive region 207 having electrically conductive particles.

As illustrated in FIGS. 2A-2C, each non-conductive material border 218A, 218B, 218A′, 218B′ may be in the form of an elongated strip with a generally rectangular shape, having an inner edge 220 and an outer edge 222. When viewed in a direction perpendicular to the front face 203 of the substrate 202, the inner edge 220 overlaps a portion of a front face 207A of the conductive adhesive region 207 of the adhesive layer 206, and the outer edge 222 extends outside the outer perimeter 217 of the conductive adhesive region 207. The inner edge 220 of the non-conductive material border 218A (FIG. 2A, 2B), 218A′ (FIG. 2C) may extend a distance 224 of at least 1 mm, at least 2 mm, at least 3 mm, or more, inward from the outer perimeter 217 of the conductive adhesive region 207. The outer edge 222 of the non-conductive material border 218A, 218A′ may extend a distance 225 of at least 1 mm, at least 2 mm, at least 3 mm, or more, outside of the outer perimeter 217 of the conductive adhesive region 207.

In an example, the non-conductive material border(s) 218A, 218B, 218A′, 218B′ may be, or may comprise, a non-conductive adhesive. The non-conductive adhesive may be a medical adhesive. The non-conductive adhesive may be sprayed onto or otherwise applied to the rest of the transducer 200 to form the non-conductive material border 218A, 218B, 218A′, 218B′. As described above, the non-conductive adhesive may be applied such that portion(s) of the outer perimeter 217 of the conductive adhesive region 207 are covered by the non-conductive adhesive. In another embodiment, the non-conductive adhesive may be applied only outside of the outer perimeter 217 of the conductive adhesive region 207, for example, starting at the outer perimeter 217 of the conductive adhesive region 207 and extending outside of the outer perimeter 217 of the conductive adhesive region 207 to form an adhesive “outline”; or starting outside the outer perimeter 217 of the conductive adhesive region 207 and extending further outside of the outer perimeter 217 of the conductive adhesive region 207 to form an adhesive “outline”. The latter approach may be advantageous compared to relying on the area of bandage outside of the outer perimeter 217 of the conductive adhesive region 207, particularly if the adhesive used for the “outline” is less irritating on the skin than the bandage adhesive. The same adhesive “outline” may be achieved in practice by coating a layer (or multiple areas) of non-conductive adhesive over portion(s) of the front face 203 of the substrate 202 prior to applying the electrode assembly comprising the conductive adhesive region 207 onto the substrate 202. In this method of construction, the layer (or multiple areas) of non-conductive adhesive extends out from beneath the conductive adhesive region 207, extending beyond the outer perimeter 217 thereby forming the adhesive “outline”.

In another example, the non-conductive material border 218A, 218B, 218A′, 218B′ may comprise a tape, bandage, or plaster. In particular, the non-conductive material border 218A, 218B, 218A′, 218B′ may comprise an electrical tape or a non-conductive medical tape. As shown in FIGS. 2B and 2C, where the conductive adhesive region 207 extends to the outer edge of the adhesive layer 206 to be covered, the conductive adhesive region 207 of the adhesive layer 206 has a front face 207A and a back face 207B, with the back face 207B facing the electrode element(s) 204. In an embodiment, for example, as shown in FIG. 2B, the non-conductive tape or bandage (218A, 218B) may adhere to the front face 207A, or on the front facing side, of the conductive adhesive region 207 within the outer perimeter 217 of the conductive adhesive region 207 and also adhere to the substrate 202 outside of the outer perimeter 217 of the conductive adhesive region 207. In another embodiment, for example, as shown in FIG. 2C, the non-conductive tape or bandage (218A′, 218B′) may adhere to the front face 207A, or on the front facing side, of the conductive adhesive region 207 within the outer perimeter 217 of the conductive adhesive region 207 and also be folded to adhere to the back face 207B, or on the back facing side, of the conductive adhesive region 207.

In an example, as shown in FIGS. 2B and 2C, the non-conductive material border 218A, 218B, 218A′, 218B′ covers a full thickness of the adhesive layer 206 (comprising the conductive adhesive region 207) in the direction perpendicular to the front face 203 of the substrate 202. In addition, as shown in FIG. 2B, the non-conductive material border 218A, 218B may be adhered to the front face 203 of the substrate 202. As such, the non-conductive material border 218A, 218B may extend from the front face 203 of the substrate 202 to the very front of the transducer 200.

As shown in FIGS. 1A-2C, the transducer (100, 200) may also include a layer of anisotropic material (108, 208) electrically coupled to the at least one electrode element (104, 204). As illustrated, the layer of anisotropic material (108, 208) is located between the electrode element(s) (104, 204) and the adhesive layer (106, 206). The layer of anisotropic material (108, 208) of FIGS. 1A-2C may be any conductive layer having different thermal and/or electrical conductivities in a direction perpendicular to the front face (103, 203) of the substrate (102, 202) than in directions that are parallel to the front face (103, 203). The layer of anisotropic material (108, 208) may be anisotropic with respect to electrical conductivity properties, anisotropic with respect to thermal properties, or both. The layer of anisotropic material may be a sheet of pyrolytic graphite, graphitized polymer film, a foil made from compressed high purity exfoliated mineral graphite, or some other material. Other details regarding layers of anisotropic materials and properties thereof are described in U.S. Patent Application Publication Nos. 2023/0037806 and 2023/0043071, which are hereby incorporated by reference in the present disclosure.

As shown in FIG. 1B, the portion(s) of the outer perimeter 116 of the adhesive layer 106 defined by the area(s) 113 devoid of electrically conductive particles may extend outward beyond an outer perimeter 128 of the layer of anisotropic material 108.

In FIGS. 1A-2C, the transducer (100, 200) may further include one or more additional electrically conductive adhesive layers. For example, the transducer (100, 200) may include a second electrically conductive adhesive layer (110, 210) located between the at least one electrode element (104, 204) and the layer of anisotropic material (108, 208). The second electrically conductive adhesive layer (110, 210) may extend from the substrate (102, 202) to the layer of anisotropic material (108, 208). Alternatively, the second electrically conductive adhesive layer (110, 210) may simply coat the front face of the at least one electrode element (104, 204) facing the layer of anisotropic material (108, 208); for example, the second adhesive layer (110, 210) may coat the dielectric layer (e.g., ceramic layer or polymer layer) of the electrode elements.

The second adhesive layer (110, 210) may have a different construction than the adhesive layer (106, 206). For example, the second adhesive layer (110, 210) may not have the same shape, size, base material, or conductive particles (e.g., fibers) that are used in the adhesive layer (106, 206). In some embodiments, the second adhesive layer (110, 210) may comprise a conductive acrylic adhesive or a conductive silicone adhesive, with or without carbon powder dispersed therein. In some embodiments, the adhesive material of the second adhesive layer (110, 210) may be the same as, or different than, the matrix material used to form the adhesive layer (106, 206).

In some embodiments, the layer of anisotropic material (108, 208) may not be present, and the transducer (100, 200) may only feature a single adhesive layer (106, 206) comprising the conductive adhesive region (107, 207) electrically coupled to the electrode element(s) (104, 204).

As constructed, transducers 100 and 200 may present exposed surfaces facing in the forward-facing direction. For the transducer 100, the forward-facing surfaces of the substrate 102 and conductive adhesive layer 107 are surfaces 130A and 130B, respectively. For the transducer 200, the forward-facing surfaces of the substrate 202, non-conductive material border 218A/B, and conductive adhesive layer 207 are surfaces 230A, 230B and 230C, respectively. The dimensions of various components of the transducer (100, 200) in FIGS. 1B, 2B, and 2C are not shown to scale, and the transducer (100, 200) may be substantially flat such that surfaces (130A-B, 230A-C) of multiple components of the transducer (100, 200) contact the subject's body upon placement of the transducer (100, 200) on the subject's body.

The disclosed adhesive layer (106, 206) comprising the conductive adhesive region (107, 207) having the area(s) 113 devoid of electrically conductive particles (alone or in combination with one or more non-conductive material borders 218A, 218B, 218A′, 218B′) may prevent or protect against a short circuit occurring between the transducer 100, 200 and an adjacent transducer positioned on a subject's body, even if one or both of the transducers have been cut. The adhesive layer (106, 206) (with or without non-conductive material border(s) 218A, 218B, 218A′, 218B′) provides a border defined by a physical barrier (e.g., area(s) 113 devoid of electrically conductive particles and/or the non-conductive material). The border surrounds an areal exclusion zone of the transducer (100, 200) containing at least the areal footprint of the second region (conductive adhesive region 107, 207) of the adhesive layer (106, 206) having the electrically conductive particles. The border (with or without non-conductive material border(s) 218A, 218B, 218A′, 218B′) may seal the outer edge of the adhesive layer (106, 206) and/or the layer of anisotropic material (108, 208) from electrical contact with other transducers in its vicinity.

The disclosed adhesive layer (106, 206) (with or without non-conductive material border(s) 218A, 218B, 218A′, 218B′) may provide a third level of separation between conductive material layer(s) of the transducer (100, 200) and a conductive portion of an adjacent transducer, in addition to (1) a recommended relative placement of the transducers on the subject's body; and (2) the non-conductive substrate (102, 202).

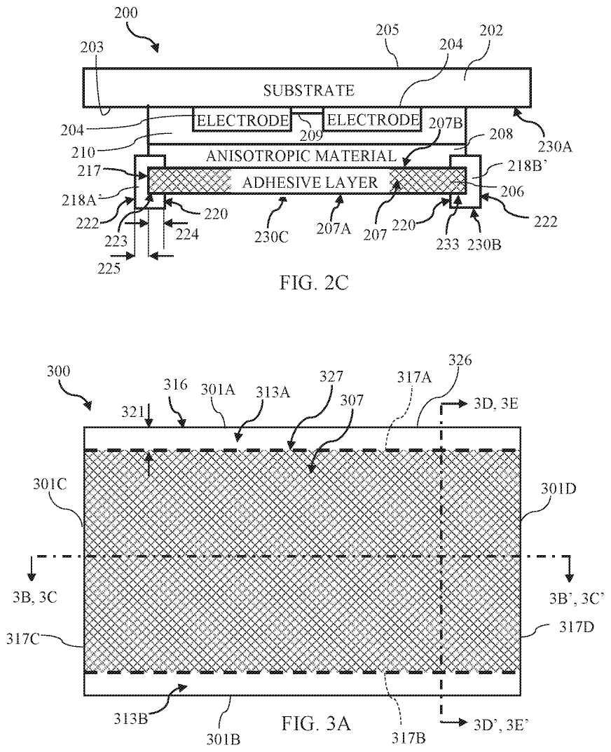

FIGS. 3A-3E depict an example adhesive layer 300 that may be used in a transducer. FIG. 3A is a bottom view showing the front face of the adhesive layer 300. FIGS. 3B and 3C are two examples of a first side cross-sectional view of the adhesive layer 300 (the cross-section as shown in FIG. 3A by the dashed line 3B, 3C-3B′, 3C′), and FIGS. 3D and 3E are two examples of a second side cross-sectional view of the adhesive layer 300 (the cross-section as shown in FIG. 3A by the dashed line 3D, 3E-3D′, 3E′).

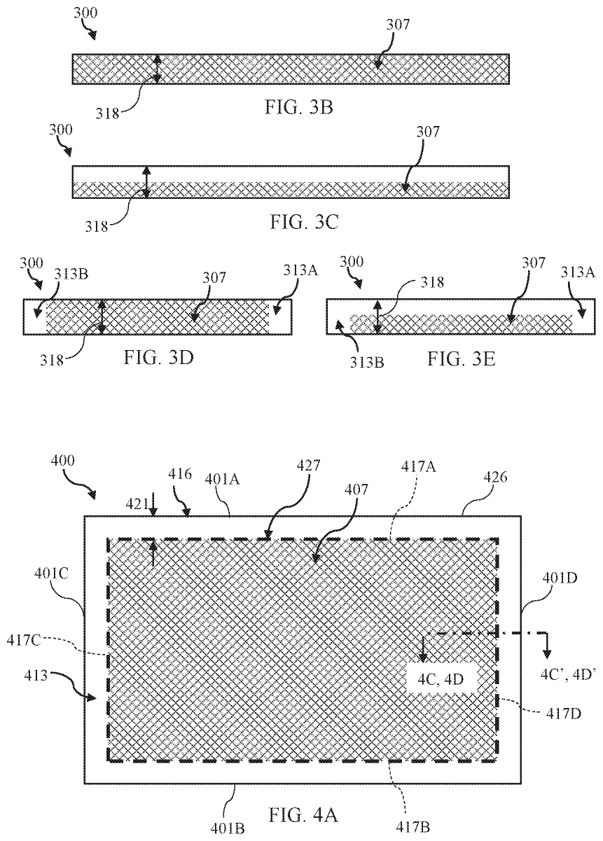

FIGS. 4A-4D depict another example adhesive layer 400 that may be used in a transducer. FIGS. 4A and 4B are bottom views of example adhesive layers 400. FIGS. 4C and 4D are two examples of a first partial cross-sectional view of the adhesive layer 400 (the cross-section as shown in FIG. 4A, FIG. 4B by the dashed line 4C, 4D-4C′, 4D′).

The adhesive layer (300, 400) of FIGS. 3A-4D may be used as the adhesive layer (106, 206) in the transducer (100, 200) of FIGS. 1A-2C. As shown in FIGS. 3A-4D, the adhesive layer (300, 400) extends in an x-y plane. The adhesive layer (300, 400) has at least one adhesive layer outer edge (301A-D, 401A-D, 401) defining an outer perimeter (316, 416) of the adhesive layer (300, 400). In an example, as shown in FIGS. 3A and 4A, the adhesive layer (300, 400) may comprise a substantially square or rectangular outer perimeter (316, 416), with or without rounded corners. As such, the adhesive layer (300, 400) may have a substantially square or rectangular shape, or substantially square or rectangular shape with rounded corners when viewed in the direction perpendicular to the x-y plane. In another example, as shown in FIG. 4B, the adhesive layer 400 may comprise a rounded outer perimeter 416. As such, the adhesive layer 400 may have a circular, oval, ovoid, ovaloid, or elliptical shape when viewed in the direction perpendicular to the x-y plane. The outer perimeter (316, 416) defines an areal footprint (326, 426) of the adhesive layer (300, 400).

In FIGS. 3A-4D, the adhesive layer (300, 400) includes a region that comprises an adhesive matrix material and a plurality of electrically conductive particles embedded at least partially within the adhesive matrix material. These electrically conductive particles embedded at least partially within the adhesive matrix material provide a conductive adhesive region (307, 407) of the adhesive layer (300, 400) illustrated via a mesh pattern in FIGS. 3A-4D.

In some embodiments, as shown in FIGS. 3B, 3D, and 4C, the plurality of electrically conductive additives forming the conductive adhesive region (307, 407) may be distributed through an entire thickness (318, 418) of the adhesive layer (300, 400) in the direction perpendicular to the x-y plane. In other embodiments, as shown in FIGS. 3C, 3E, and 4D, the plurality of electrically conductive additives forming the conductive adhesive region (307, 407) may be distributed through only a portion of the entire thickness (318, 418) of the adhesive layer (300, 400) in the direction perpendicular to the x-y plane. This portion of the entire thickness (318, 418) may be substantially towards one face of the adhesive layer (300, 400) as shown in FIGS. 3C, 3E, and 4D or may be embedded within the adhesive layer (300, 400).

In FIGS. 3A-4D, the adhesive layer (300, 400) includes at least one non-conductive edge portion (313A/B, 413) having the adhesive matrix material devoid of the electrically conductive particles. The at least one non-conductive edge portion (313A/B, 413) is electrically non-conductive. When viewed in a direction perpendicular to the x-y plane (e.g., FIGS. 3A, 4A, and 4B), at least one non-conductive edge portion (313A/B, 413) of the adhesive layer (300, 400) extends along an outer perimeter (317A/B, 417A-D, 417) of the conductive adhesive region (307, 407) and forms at least a portion of the outer perimeter (316, 416) of the adhesive layer (300, 400). This non-conductive edge portion (313A/B, 413) may extend a distance (321, 421) of at least 1 mm, at least 2 mm, at least 3 mm, or more, from the adhesive layer outer edge (301A/B, 401A-D, 401) into the adhesive layer (300, 400) in a direction perpendicular to the adhesive layer outer edge (301A/B, 401A-D, 401).

Turning to FIG. 3A, the non-conductive edge portion(s) 313A/B may not extend along the entire outer perimeter 316 of the adhesive layer 300. For example, the adhesive layer 300 may comprise a first non-conductive edge portion 313A extending along a first outer edge 317A of the conductive adhesive region 307, and a second non-conductive edge portion 313B extending along a second outer edge 317B of the conductive adhesive region 307 opposite the first outer edge 317A of the conductive adhesive region 307. As shown, the first and second non-conductive edge portions 313A and 313B are separated from each other. As such, electrically conductive particles are located along a third outer edge 317C of the conductive adhesive region 307 connecting the first and second outer edges 317A/B of the conductive adhesive region 307. Similarly, electrically conductive particles are located along a fourth outer edge 317D of the conductive adhesive region 307 connecting the first and second outer edges 317A/B of the conductive adhesive region 307.

Turning to FIGS. 4A and 4B, the non-conductive edge portion 413 may extend along the entire outer perimeter 416 of the adhesive layer 400. As shown in FIG. 4A, for example, the non-conductive edge portion 413 may extend along four outer edges 401A-D of the adhesive layer 400, the four outer edges 401A-D being connected by corners or rounded corners. As shown in FIG. 4B, the non-conductive edge portion 413 may extend along an entire rounded outer edge 401 of the adhesive layer 400.

In an example, the plurality of electrically conductive particles may be electrically conductive fibers. As such, the conductive adhesive region (307, 407) may be a “fiber region” having conductive carbon fibers, and the non-conductive edge portion(s) (313A/B, 413) may be “non-fiber regions” each devoid of electrically conductive fibers. When viewed in a direction perpendicular to the x-y plane (e.g., FIGS. 3A, 4A, and 4B), the plurality of electrically conductive fibers are located within the fiber region (307, 407) of the adhesive layer (300, 400) defined by a first areal footprint (327, 427).

The non-fiber region(s) (313A/B, 413) are located along one or more portions (317A/B, 417A-D, 417) of an outer perimeter of the fiber region (307, 407) of the adhesive layer (300, 400). For example, in FIG. 3A, the adhesive layer (300, 400) may include: a first non-fiber region 313A located along a first portion 317A of the outer perimeter of the fiber region 307; and a second non-fiber region 313B located along a second portion 317B of the outer perimeter of the fiber region 307 opposite the first portion 317A. A third portion 317C of the outer perimeter of the fiber region 307 may form a portion 301C of the outer perimeter 316 of the adhesive layer 300; and a fourth portion 317D of the outer perimeter of the fiber region 307 may form another portion 301D of the outer perimeter 316 of the adhesive layer 300. As another example, shown in FIGS. 4A and 4B, the adhesive layer 400 may comprise one non-fiber region 413 fully surrounding the first areal footprint 427 of the fiber region 407.

The plurality of electrically conductive particles may comprise graphite. The plurality of electrically conductive particles may comprise a sheet of fibers embedded in the adhesive matrix material. The sheet of fibers may be in the form of a mesh layer that can be cut to any desired shape, which becomes the first areal footprint (327, 427) of the conductive adhesive region (307, 407). The electrically conductive fibers may be oriented such that the longitudinal axes of each of the fibers is substantially (e.g., within 10 degrees) parallel to the x-y plane of the adhesive layer (300, 400). The adhesive matrix material may comprise any suitable polymer, for example, the adhesive matrix material may comprise an acrylic polymer matrix material or a silicone polymer matrix material. In some embodiments where the non-fiber region comprises a non-conductive adhesive layer, the matrix material (e.g., adhesive) may be the same in each of the non-conductive region (non-fiber region) and the conductive adhesive region (fiber region). In other embodiments of this aspect, the matrix material (e.g., adhesive) may not be the same in each of the non-conductive region (non-fiber region) and the conductive adhesive region (fiber region).

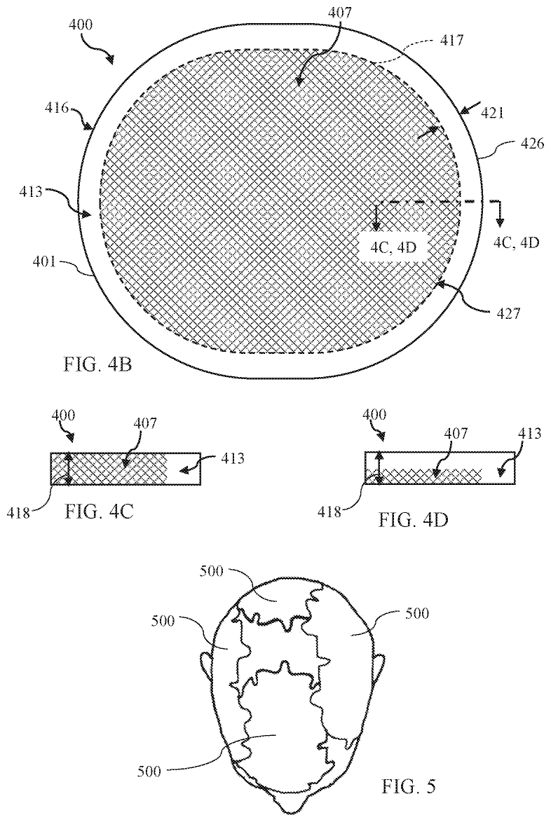

FIG. 5 depicts an example arrangement of transducers 500 located on a subject's head. FIG. 5 depicts an example of a subject's head on which transducers 500 are placed in varying positions and/or orientations. Such arrangements of transducers 500 on a subject's head are capable of applying TTFields to a tumor in a region of the subject's brain. The transducers 500 illustrated in FIG. 5 have different shapes than the transducers 100, 200 shown in the embodiments of FIGS. 1A and 2A. In particular, the transducers 500 include tabs and recesses along an outer perimeter of the transducer substrate, giving the outer perimeter a scalloped edge. However, transducers having straight or more uniformly curved edges on the outer perimeter of the transducer substrate, such as shown in FIGS. 1A and 2A, may be arranged on the subject's head in a similar manner as the transducers 500 of FIG. 5. In addition, the substrate (102, 202) of FIGS. 1A and 2A may, in other embodiments, have a scalloped outer perimeter as shown in FIG. 5.

As illustrated, some of the adjacent transducers 500 may overlap each other on the subject's head. A user may cut one or more portions of the transducer(s) 500 to fit the transducers 500 together, to fit the transducers 500 around anatomical features, or simply to reduce the amount of adhesive touching the subject's body. The transducers 500 may be equipped with one or more of the above-described electrically conductive adhesive layers having region(s) devoid of electrically conductive particles (e.g., FIGS. 1A-2C). For transducers positioned on the head, or, indeed, for transducers positioned anywhere else on the body, these protective borders may provide an additional layer of protection against a short circuit.

### ILLUSTRATIVE EMBODIMENTS

The invention includes other illustrative embodiments (“Embodiments”) as follows.

Embodiment 1: An adhesive layer for use in a transducer apparatus, the adhesive layer extending in an x-y plane and having an adhesive layer outer edge, the adhesive layer comprising: an adhesive matrix material; a plurality of electrically conductive particles embedded at least partially within the adhesive matrix material forming a conductive adhesive region of the adhesive layer; and at least one non-conductive edge portion comprising an adhesive devoid of electrically conductive particles, the at least one non-conductive edge portion being electrically non-conductive; wherein, when viewed in a direction perpendicular to the x-y plane, a first non-conductive edge portion is located adjacent to and extends along an outer edge of the conductive adhesive region and forms at least a portion of an outer perimeter of the adhesive layer.

Embodiment 2: The adhesive layer of Embodiment 1, wherein the first non-conductive edge portion extends at least 1 mm from the adhesive layer outer edge into the adhesive layer in a direction perpendicular to the adhesive layer outer edge.

Embodiment 3: The adhesive layer of Embodiment 1, wherein the adhesive layer has a substantially square or rectangular shape, or substantially square or rectangular shape with rounded corners when viewed in the direction perpendicular to the x-y plane.

Embodiment 4: The adhesive layer of Embodiment 3, wherein, when viewed in the direction perpendicular to the x-y plane, the conductive adhesive region has a substantially square or rectangular shape, or substantially square or rectangular shape with rounded corners, and the first non-conductive edge portion is located adjacent to and extends along four outer edges of the conductive adhesive region, the four outer edges being connected by corners or rounded corners.

Embodiment 5: The adhesive layer of claim 3, wherein, when viewed in the direction perpendicular to the x-y plane, the conductive adhesive region has a substantially square or rectangular shape, or substantially square or rectangular shape with rounded corners, and the first non-conductive edge portion is located adjacent to and extends along a first outer edge of the conductive adhesive region, and a second non-conductive edge portion is located adjacent to and extends along a second outer edge of the conductive adhesive region opposite the first outer edge of the conductive adhesive region, each forming at least a portion of the outer perimeter of the adhesive layer.

Embodiment 6: The adhesive layer of Embodiment 5, wherein, when viewed in the direction perpendicular to the x-y plane, the first and second non-conductive edge portions are separated from each other, such that electrically conductive particles are located along a third outer edge of the conductive adhesive region connecting the first and second outer edges of the conductive adhesive region, and electrically conductive particles are located along a fourth outer edge of the conductive adhesive region connecting the first and second outer edges of the conductive adhesive region.

Embodiment 7: The adhesive layer of Embodiment 1, wherein the adhesive layer has a circular, oval, ovoid, ovaloid, or elliptical shape when viewed in the direction perpendicular to the x-y plane.

Embodiment 8: The adhesive layer of Embodiment 7, wherein, when viewed in the direction perpendicular to the x-y plane, the first non-conductive edge portion is located adjacent to and extends along the outer edge of the conductive adhesive region and forms the entire outer perimeter of the adhesive layer.

Embodiment 9: The adhesive layer of Embodiment 1, wherein, when viewed in the direction perpendicular to the x-y plane, the first non-conductive edge portion is located adjacent to and extends along the outer edge of the conductive adhesive region and forms the entire outer perimeter of the adhesive layer.

Embodiment 10: The adhesive layer of Embodiment 1, wherein the plurality of electrically conductive particles are fibers.

Embodiment 11: The adhesive layer of Embodiment 1, wherein the plurality of electrically conductive particles comprises graphite.

Embodiment 12: The adhesive layer of Embodiment 1, wherein the plurality of electrically conductive particles comprises a sheet of fibers embedded in the adhesive matrix material.

Embodiment 13: The adhesive layer of Embodiment 1, wherein the plurality of electrically conductive particles is distributed through an entire thickness of the adhesive layer in the direction perpendicular to the x-y plane.

Embodiment 14: The adhesive layer of Embodiment 1, wherein the plurality of electrically conductive particles is distributed through a portion of an entire thickness of the adhesive layer in the direction perpendicular to the x-y plane.

Embodiment 15: A transducer apparatus for delivering tumor treating fields to a subject's body, the transducer apparatus comprising: a substrate; at least one electrode element coupled to the substrate; and an adhesive layer located on an opposite side of the at least one electrode element from the substrate, the adhesive layer comprising an electrically conductive adhesive region, wherein: the electrically conductive adhesive region has an areal footprint that overlays one, or more than one, electrode element; the electrically conductive adhesive region comprises a plurality of electrically conductive fibers embedded at least partially within an adhesive matrix material, and when viewed in a direction perpendicular to a face of the substrate, a first area located adjacent to and extending along at least a first portion of an outer perimeter of the electrically conductive adhesive region is devoid of electrically conductive fibers, is electrically non-conductive, and forms at least a first portion of an outer perimeter of the adhesive layer.

Embodiment 16: The transducer apparatus of Embodiment 15, wherein, when viewed in the direction perpendicular to the face of the substrate, a second area defining a second portion of an outer perimeter of the adhesive layer has electrically conductive fibers, and the transducer apparatus further comprises a non-conductive material border disposed over the second area, the non-conductive material border being electrically non-conductive.

Embodiment 17: The transducer apparatus of Embodiment 16, wherein, when viewed in a direction parallel to the face of the substrate, the non-conductive material border covers a full thickness of the adhesive layer in the direction perpendicular to the face of the substrate.

Embodiment 18: The transducer apparatus of Embodiment 16, wherein, when viewed in the direction perpendicular to the face of the substrate, an outer edge of the non-conductive material border extends at least 1 mm outside of the second portion of the outer perimeter of the adhesive layer.

Embodiment 19: The transducer apparatus of Embodiment 16, wherein, when viewed in the direction perpendicular to the face of the substrate, a third area defining a third portion of the outer perimeter of the adhesive layer opposite the second portion of the outer perimeter has electrically conductive fibers, and a second non-conductive material border is disposed over the third area, the second non-conductive material border being electrically non-conductive.

Embodiment 20: The transducer apparatus of Embodiment 16, wherein the non-conductive material border comprises a non-conductive adhesive.

Embodiment 21: The transducer apparatus of Embodiment 16, wherein the non-conductive material border comprises a tape, bandage, or plaster.

Embodiment 22: The transducer apparatus of Embodiment 16, wherein the non-conductive material border comprises a tape, bandage or plaster, wherein the tape, bandage or plaster adheres to a front face of the adhesive layer and is folded to adhere to a back face, or on a back facing side, of the adhesive layer.

Embodiment 23: The transducer apparatus of Embodiment 15, wherein, when viewed in the direction perpendicular to the face of the substrate, the first area located adjacent to and extending along at least the first portion of the outer perimeter of the electrically conductive adhesive region extends along the entire outer perimeter of the electrically conductive adhesive region, and forms an entire outer perimeter of the adhesive layer that is non-conductive.

Embodiment 24: The transducer apparatus of Embodiment 15, further comprising a layer of anisotropic material located between one or more than one electrode element and the electrically conductive adhesive region.

Embodiment 25: The transducer apparatus of Embodiment 24, wherein, when viewed in the direction perpendicular to the face of the substrate, the first portion of the outer perimeter of the adhesive layer that is electrically non-conductive extends outward beyond an outer perimeter of the layer of anisotropic material. Embodiment 26: The transducer apparatus of Embodiment 24, further comprising a second adhesive layer located between the at least one electrode element and the layer of anisotropic material.

Embodiment 27: The transducer apparatus of Embodiment 15, wherein the at least one electrode element comprises a ceramic dielectric layer.

Embodiment 28: The transducer apparatus of Embodiment 15, wherein the at least one electrode element comprises a polymer film.

Embodiment 29: An adhesive layer for use in a transducer apparatus, the adhesive layer extending in an x-y plane, the adhesive layer comprising: an adhesive matrix material; and a plurality of electrically conductive fibers embedded at least partially within the adhesive matrix material, wherein, when viewed in a direction perpendicular to the x-y plane, the plurality of electrically conductive fibers are located within a fiber region of the adhesive layer defined by a first areal footprint, and the adhesive layer further comprises at least one non-fiber region located along one or more portions of an outer perimeter of the fiber region of the adhesive layer, each non-fiber region being devoid of electrically conductive fibers.

Embodiment 30: The adhesive layer of Embodiment 29, wherein, when viewed in the direction perpendicular to the x-y plane, the adhesive layer comprises one non-fiber region fully surrounding the first areal footprint.

Embodiment 31: The adhesive layer of Embodiment 29, wherein, when viewed in the direction perpendicular to the x-y plane, the adhesive layer comprises: a first non-fiber region located along a first portion of the outer perimeter of the fiber region of the adhesive layer; and a second non-fiber region located along a second portion of the outer perimeter of the fiber region of the adhesive layer opposite the first portion.

Embodiment 32: The adhesive layer of Embodiment 31, wherein, when viewed in the direction perpendicular to the x-y plane: a third portion of the outer perimeter of the fiber region of the adhesive layer forms a portion of an outer perimeter of the adhesive layer; and a fourth portion of the outer perimeter of the fiber region of the adhesive layer forms another portion of the outer perimeter of the adhesive layer.

Embodiment 33: The adhesive layer of Embodiment 29, wherein, when viewed in the direction perpendicular to the x-y plane, the adhesive layer comprises a rounded outer perimeter.

Embodiment 34: The adhesive layer of Embodiment 29, wherein, when viewed in the direction perpendicular to the x-y plane, the adhesive layer comprises a substantially square or rectangular outer perimeter, with or without rounded corners.

Embodiment 35: The adhesive layer of Embodiment 1, wherein the adhesive in the at least one non-conductive edge portion is the same as the adhesive matrix material in the conductive adhesive region.

Embodiment 36: The adhesive layer of Embodiment 1, wherein the adhesive in the at least one non-conductive edge portion is not the same as the adhesive matrix material in the conductive adhesive region.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. An adhesive layer for use in a transducer apparatus, the adhesive layer extending in an x-y plane and having an adhesive layer outer edge, the adhesive layer comprising:
an adhesive matrix material;
a plurality of electrically conductive particles embedded at least partially within the adhesive matrix material forming a conductive adhesive region of the adhesive layer; and
at least one non-conductive edge portion comprising an adhesive devoid of electrically conductive particles, the at least one non-conductive edge portion being electrically non-conductive;
wherein, when viewed in a direction perpendicular to the x-y plane,
a first non-conductive edge portion is located adjacent to and extends along an outer edge of the conductive adhesive region and forms at least a portion of an outer perimeter of the adhesive layer.

2. The adhesive layer of claim 1, wherein the first non-conductive edge portion extends at least 1 mm from the adhesive layer outer edge into the adhesive layer in a direction perpendicular to the adhesive layer outer edge.

3. The adhesive layer of claim 1, wherein the adhesive layer has a substantially square or rectangular shape, or substantially square or rectangular shape with rounded corners when viewed in the direction perpendicular to the x-y plane.

4. The adhesive layer of claim 3, wherein, when viewed in the direction perpendicular to the x-y plane,
the conductive adhesive region has a substantially square or rectangular shape, or substantially square or rectangular shape with rounded corners, and the first non-conductive edge portion is located adjacent to and extends along four outer edges of the conductive adhesive region, the four outer edges being connected by corners or rounded corners.

5. The adhesive layer of claim 3, wherein, when viewed in the direction perpendicular to the x-y plane,
the conductive adhesive region has a substantially square or rectangular shape, or substantially square or rectangular shape with rounded corners, and the first non-conductive edge portion is located adjacent to and extends along a first outer edge of the conductive adhesive region, and a second non-conductive edge portion is located adjacent to and extends along a second outer edge of the conductive adhesive region opposite the first outer edge of the conductive adhesive region, each forming at least a portion of the outer perimeter of the adhesive layer.

6. The adhesive layer of claim 5, wherein, when viewed in the direction perpendicular to the x-y plane,
the first and second non-conductive edge portions are separated from each other, such that electrically conductive particles are located along a third outer edge of the conductive adhesive region connecting the first and second outer edges of the conductive adhesive region, and electrically conductive particles are located along a fourth outer edge of the conductive adhesive region connecting the first and second outer edges of the conductive adhesive region.

7. The adhesive layer of claim 1, wherein the adhesive layer has a circular, oval, ovoid, ovaloid, or elliptical shape when viewed in the direction perpendicular to the x-y plane.

8. The adhesive layer of claim 1, wherein, when viewed in the direction perpendicular to the x-y plane,
the first non-conductive edge portion is located adjacent to and extends along the outer edge of the conductive adhesive region and forms the entire outer perimeter of the adhesive layer.

9. The adhesive layer of claim 1, wherein the plurality of electrically conductive particles are fibers.

10. The adhesive layer of claim 1, wherein the plurality of electrically conductive particles comprise graphite.

11. The adhesive layer of claim 1, wherein the plurality of electrically conductive particles comprises a sheet of fibers embedded in the adhesive matrix material.

12. A transducer apparatus for delivering tumor treating fields to a subject's body, the transducer apparatus comprising:
a substrate;
at least one electrode element coupled to the substrate; and
an adhesive layer located on an opposite side of the at least one electrode element from the substrate, the adhesive layer comprising an electrically conductive adhesive region, wherein:
the electrically conductive adhesive region has an areal footprint that overlays one, or more than one, electrode element;
the electrically conductive adhesive region comprises a plurality of electrically conductive fibers embedded at least partially within an adhesive matrix material, and
when viewed in a direction perpendicular to a face of the substrate, a first area located adjacent to and extending along at least a first portion of an outer perimeter of the electrically conductive adhesive region is devoid of electrically conductive fibers, is electrically non-conductive, and forms at least a first portion of an outer perimeter of the adhesive layer.

13. The transducer apparatus of claim 12, wherein,
when viewed in the direction perpendicular to the face of the substrate, a second area defining a second portion of the outer perimeter of the adhesive layer has electrically conductive fibers, and
the transducer apparatus further comprises a non-conductive material border disposed over the second area, the non-conductive material border being electrically non-conductive.

14. The transducer apparatus of claim 13, wherein, when viewed in a direction parallel to the face of the substrate,
the non-conductive material border covers a full thickness of the adhesive layer in the direction perpendicular to the face of the substrate.

15. The transducer apparatus of claim 13, wherein, when viewed in the direction perpendicular to the face of the substrate,
an outer edge of the non-conductive material border extends at least 1 mm outside of the second portion of the outer perimeter of the adhesive layer.

16. The transducer apparatus of claim 13, wherein,
when viewed in the direction perpendicular to the face of the substrate, a third area defining a third portion of the outer perimeter of the adhesive layer opposite the second portion of the outer perimeter has electrically conductive fibers, and
a second non-conductive material border is disposed over the third area, the second non-conductive material border being electrically non-conductive.

17. The transducer apparatus of claim 12, wherein, when viewed in the direction perpendicular to the face of the substrate,
the first area located adjacent to and extending along at least the first portion of the outer perimeter of the electrically conductive adhesive region extends along the entire outer perimeter of the electrically conductive adhesive region, and forms an entire outer perimeter of the adhesive layer that is non-conductive.

18. The transducer apparatus of claim 12, further comprising a layer of anisotropic material located between one or more than one electrode element and the electrically conductive adhesive region.

19. The transducer apparatus of claim 18, wherein, when viewed in the direction perpendicular to the face of the substrate,
the first portion of the outer perimeter of the adhesive layer that is electrically non-conductive extends outward beyond an outer perimeter of the layer of anisotropic material.

20. An adhesive layer for use in a transducer apparatus, the adhesive layer extending in an x-y plane, the adhesive layer comprising:
an adhesive matrix material; and
a plurality of electrically conductive fibers embedded at least partially within the adhesive matrix material,
wherein, when viewed in a direction perpendicular to the x-y plane,
the plurality of electrically conductive fibers are located within a fiber region of the adhesive layer defined by a first areal footprint, and
the adhesive layer further comprises at least one non-fiber region located along one or more portions of an outer perimeter of the fiber region of the adhesive layer, each non-fiber region being devoid of electrically conductive fibers.
